(19) United States
(12) Patent Application Publication
(10) Pub. No.: US 2018/0343815 A1
(43) Pub. Date: Dec. 6, 2018

(54) PRUNING METHOD FOR IMPROVED SEED PRODUCTION

(71) Applicant: NUNHEMS B.V., Nunhem (NL)

(72) Inventor: Matthias THOLEN, Nunhem (NL)

(21) Appl. No.: 15/555,268

(22) PCT Filed: Mar. 7, 2016
(86) PCT No.: PCT/EP2016/054769

§ 371 Date: Sep. 1, 2017

(30) Foreign Application Priority Data
Mar. 6, 2015 (EP) 15158025.5

(51) Int. Cl. A01H 1/02; A01G 22/00; A01G 7/04

(52) U.S. Cl. CPC A01H 1/02; A01G 7/04; A01G 22/00

(57) ABSTRACT

The present invention relates to a method for pruning pepper plants. In particular, pepper plants are pruned to a specific branch-architecture, whereby seed yield is increased.

### FIELD OF THE INVENTION

The present invention relates to the field of pepper seed production. More specifically, the invention relates to an improved method for producing seed of Capsicum, characterized in that the female parent plant producing the seed has been pruned to a specific number of branches, such as 3, 4, or 5 branches or even 6, 7 or 8 branches.

### BACKGROUND

The present invention relates to the field of pepper seed production, i.e. to the production of seed of Capsicum. The genus Capsicum belongs to the Solanaceae family. The Solanacea family includes more than 2600 different species including tomato (Solanum lycopersicum), potato (Solanum tuberosum), and pepper (Capsicum species) as best known species.

The goal of vegetable breeding is to combine various desirable traits in a single variety. Such desirable traits may include greater yield, resistance to insects or pests, tolerance to heat and drought, better agronomic quality, higher nutritional value, growth rate and/or fruit properties.

It is appreciated in the field that an increase in flower density results in a decrease in fruit and seed yield due to e.g., abortion of fruits and slow fruit development as a result of source competition (e.g., referred is to Marcelis, 2004). This knowledge is the basis for pruning of pepper plants in greenhouses. By restricting the number of branches, the canopy light interception, fruit set and fruit quality is improved (e.g., referred is to Jovicich et al., 2004). Widely applied in pepper fruit production is the so called “two-stem pruning architecture”, or “V” trellis system in pepper fruit production. Pepper plants pruned to “V” trellis system have been pruned by removing one of the two shoots that develop at each node except for the first node formed on the stem, resulting in a plant with two branches on top of the stem. The two branches are in general supported vertically with twine. The “V” trellis system is used by Canadian and Dutch greenhouse grower and has been adopted by many Florida Greenhouse growers (Jovicich et al., 2004).

### DESCRIPTION

### DETAILED DESCRIPTION OF THE INVENTION

Invented is an improved method for pepper seed production, which was found to result in up to 160% increase in seed yield. The method is characterized in that a pruning architecture of 3, 4, 5, 6, 7, or even 8 branches per plant is applied to the seed producing female parent plant.

The invention provides a method for producing seeds of Capsicum, comprising the steps:
a) providing a first Capsicum parent plant;
b) pruning the plant to a specific number of branches, selected from 3, 4, 5, 6, 7 or 8 branches;
c) emasculating flowers produced on said plant;
d) allowing pollination of said flowers with the pollen of a second Capsicum parent plant;
e) allowing fruits to grow; and optionally,
f) harvesting said fruits and collecting seeds from the fruits of e).

In one aspect the method further comprises optionally the step g) separating the mature seeds from the less mature seeds and/or immature seeds.

Optionally, the method of the invention further comprising the step of
h) treating the harvested seeds of step f) or the mature seed of step g).

The method results in an average mature seed yield per plant being increased by at least 10% compared to the average mature seed yield per plant when the same method is applied under the same conditions with the exception that first plant under step a) is pruned to the conventional 2-branch architecture in step b).

Treating the seed includes, but is not limited to, one or more of the following: cleaning, washing, drying, hydrating, disinfection, selection for viability, seed priming, seed coating (e.g. film coating), seed pelleting as defined herein, adding a seed treatment formulation (such as a composition comprising at least one insecticidal, a fungicidal, acaricidal or nematicidal compound, or a plant growth regulator or a biological control agent), and/or combinations of such treatments. For example, in one aspect the seeds of the plant may in a first step be hydrated, in a second step be dried and in a third step be treated with a seed treatment formulation. Methods of treating the seed are disclosed for instance in WO2008/107097A1, which is incorporated herein by reference.

It is understood that the methods described herein are generally applied to a plurality of plants, e.g. in step a) a plurality of plants is provided, e.g. at least 10, 20, 30, 40, 50, 60, 70, 80, 100, 500, 1000, 2000, 3000, 4000, 5000, 6000, 7000, 8000, 9000, 10000 or more plants. It is understood that the plurality of plants are of the same genotype, so when e.g. referring to a plurality of plants of an inbred parent line all plants are plants of the same inbred parent line. So in step a) in one aspect all plants are of the same parent line, preferably the same inbred parent line. Likewise, it is understood that the pollen of the second parent plant in step d) is pollen of one genotype, e.g. pollen of a parent line, e.g. a parent inbred line.

The first and second Capsicum parent plants of the method of the invention, also denominate herein as first and second parent plants, and the seed of Capsicum of the method of the invention, also denominated herein as seeds, are understood to be plants and seeds that belong to the genus Capsicum, preferably to the same species of domesticated pepper, most preferably the same species of cultivated pepper. In one aspect both parents are cultivated pepper of the species Capsicum annuum, such as inbred lines of C. annuum, and the resulting seed is seed of an F1 hybrid, especially an F1 hybrid cultivated pepper variety.

In one aspect the F1 hybrid produces fruits of a particular type, i.e. the parent lines are inbred lines which produce a particular fruit type, preferably the same fruit type. Fruit types include for example Bell (blocky), Lamuyo type (long blocky), Dulce Italiano, Dulce California, Dulce Hungario, snack type pepper, conical, capia, sweet charleston, dolma, cherry, jalapeno, shakira, pencil or hot Charleston, Sivri (e.g. Demre Sivri or Kazanli Sivri), Hungarian wax, Kapya/Florinis, banana, Fresno, Serrano, Ancho, Anaheim, Pasilla, Santa Fe, Scotch bonnet, Habanero. Some exemplary but non-limiting pictures of different types of pepper were disclosed in Bosland et al. (1996). The fruits may also be of any color. The fruits may be seedless or seeded.

Thus in one aspect, the parent plants and seed of the method of the invention are seed of a Capsicum annuum, such as sweet or hot pepper of different fruit types, e.g. Bell (blocky), Lamuyo type (long blocky), Dulce Italiano, Dulce California, Dulce Hungario, snack type pepper, conical, capia, sweet charleston, dolma, cherry, jalapeno, shakira, pencil or hot Charleston, Sivri (e.g. Demre Sivri or Kazanli Sivri), Hungarian wax, Kapya/Florinis, banana, Fresno, Serrano, Ancho, Anaheim, Pasilla, Santa Fe, Scotch bonnet, Habanero. In one embodiment, the parent plants and seed of the method of the invention are plants and seed of a sweet pepper, of the species Capsicum annum such as, but not limited to, Bell pepper, Lamuyo type, Dulce Italiano, Dulce Italiano, Dulce California, Dulce Hungario, snack type pepper, Capia, conical, sweet Charleston, Dolma; or hot pepper, of the species Capsicum annum such as, but not limited to Jalapeno, or conico types.

Thus, in one aspect in step a) of the method a first parent plant is provided, preferably a plurality of plants, and preferably the first parent plant is an inbred line of cultivated pepper, most preferably an inbred line of cultivated sweet pepper. This first parent plant is pruned to a specific number of branches selected from 3, 4, 5, 6, 7 or 8 branches, flowers are emasculated and pollination is allowed in step d), preferably with pollen of a second parent plant. This second parent plant is preferably an inbred line of cultivated pepper, most preferably an inbred line of cultivated sweet pepper, so that the resulting seeds are F1 hybrid seeds of cultivated pepper, C. annuum, preferably of cultivated sweet pepper.

In another aspect both parents are cultivated pepper of a species selected from Capsicum chinense, Capsicum frutescens, Capsicum baccatum and Capsicum pubescens, such as inbred lines of the species, and the resulting seed is seed of an F1 hybrid, especially an F1 hybrid cultivated pepper variety.

In the method of the invention, the first parent plant is emasculated in step c). The first parent plant is therefore also referred to as the female parent plant. In the method of the invention, pollen used in step d) is derived from the second parent plant. The second parent plant is therefore also referred to as the male parent plant. As mentioned, these are in one aspect preferably both inbred lines of cultivated pepper, also referred to as inbred parent lines.

Thus the first and/or second parent plant of the method of the invention may be an inbred line. In one aspect both parent plants of the method of the invention are inbred lines.

The first parent plant of step a) in the method of the invention may be a young pepper plant, or seedling, e.g. a plant that has been raised for a number of weeks, e.g. 6, 7, 8 or more weeks after sowing. The first parent may for example be raised in a nursery, such as a suitable nursery for raising pepper plants as known by the skilled person (e.g., referred is to Jovicich et al., 1999; Maboko and Chiloane, 2012) and may then be transplanted into a greenhouse, where the plant is then pruned to a specific number of branches during its further growth. The term “obtaining a first parent plant” in step a) of the method of the invention may encompass sowing a first parent plant (e.g. in a nursery), raising it for several weeks and transplanting said plant to a greenhouse. Alternatively, the term “obtaining a first parent plant” in step a) may encompass obtaining young pepper plants which are large enough for growth in a greenhouse. In one aspect of the invention, the pepper plants of step a) comprise the stem with the first 7 nodes, comprising a leaf at each node, and the first splitting at node 7 as shown in FIG. 1.

Pepper has a specific way of growing. The stem grows and after about 7 leaves are formed, two growing points (heads) are formed (which grow out into two branches). This is called the ‘first splitting’ (which is thereafter referred to as node zero) and each of these two growing points grows into a ‘shoot’ or ‘branch’, see FIG. 1. Each node that forms when these two branches grow further will again form two growing points, and so on. The node of the first splitting is numbered as zero, from which each new nodes is counted upwards. Typically, a “V” trellis architecture or 2-branch architecture known in the field is reached by keeping the two shoots of the first splitting and removing one of the two heads (or growing points) of each new node formed after the first splitting.

In the instant invention however heads (or shoots) are removed in such a way that 3, 4, 5, 6, 7 or even 8 branches are formed. This is done by pruning the plant during its growth, i.e. by removing the supernumerary heads (or shoots) at the newly formed nodes, to result in a plant having a specific number of branches, selected from 3 branches, 4, 5, 6, 7 or even 8 branches.

So for example to keep 3 branches only, one head on the 2-branch plant is not removed but allowed to grow (while at all other nodes of the branch one head is removed). On that new 3^{rd} branch, at each new node one of the two heads is removed to avoid further branches forming. See FIG. 1, where the 3^{rd} branch is allowed to grow from node 1. It can equally be allowed to grow from another node of the branch, such as node 2 or 3, or a later node.

Similarly to keep 4, 5, 6, 7 or 8 branches, the supernumerary heads are removed. FIG. 2 shows a plant that is pruned to a 4-branch architecture by allowing one head on each of the first branches to grow out, in this example from node 1 of each branch, but also other nodes could be left on the plant to grow out, such as node 2 or 3 of each branch.

Similarly, the 3^{rd} and 4^{th} branch could also be left to grow from the same first branch and/or the 4^{th} branch could grow from the 3^{rd} branch. However preferably the weaker inward facing branches are removed during pruning and it is also desired to achieve a final architecture where the branches are relatively evenly distributed when looking at the plant from above, as shown in FIG. 3. So in one aspect it is preferred that in a 4-branch plant, the 3^{rd} and 4^{th} branch are attached to nodes 1 (as shown in FIG. 2), 2 or 3 of the first and second branch.

Removal of the heads or shoots (pruning) can be done as soon as the heads have formed, but can also be done at a later developmental stage when a shoot or branch has already formed from the head/growing point. Thus, when referring to removal of heads anywhere herein, this encompasses removal of shoots or branches developed from the heads (and vice versa, thus pruning of heads and shoots, or branches, are used interchangeably herein). However, early removal is desired to avoid the plant spending energy resources on shoot or branch growth.

Pruning to a specific number of branches, also designated as x branches, wherein x is selected from 3, 4, 5, 6, 7 or 8, is in one aspect performed by allowing the plant to grow past the first splitting (indicated as node ‘0’ in FIGS. 1 and 2) and allowing x branches to form by removing one of the two heads (or growing points) of each new splitting (referred to herein as pruned splitting) other than the un-pruned splitting(s) from which the specific branches grow. So, in FIG. 1, there is one un-pruned splitting following the first splitting, while all other splittings are pruned by removing one of the two heads or shoots. In FIG. 2 there are two un-pruned splittings following the first splitting, while all other splittings are pruned. Thus, to arrive at a 3-branch architecture 1 splitting after the first splitting is left unpruned (while all other splittings are pruned); to arrive at a 4-branch architecture 2 splittings after the first splitting is left unpruned (while all other splittings are pruned); to arrive at a 5-branch architecture 3 splittings after the first splitting is left unpruned (while all other splittings are pruned); to arrive at a 6-branch architecture 4 splittings after the first splitting is left unpruned (while all other splittings are pruned); to arrive at a 7-branch architecture 5 splittings after the first splitting is left unpruned (while all other splittings are pruned); and to arrive at a 8-branch architecture 6 splittings after the first splitting is left unpruned (while all other splittings are pruned). In all branch architectures disclosed herein preferably the weakest head or weakest shoot is removed. Mostly, the weakest head or shoot is an inward facing head or shoot. However, in case an outward facing head or shoot is weaker as compared to an inward facing head or shoot, such weaker outward facing head or shoot may be removed first.

In one aspect pruning is done in such a way that the adult plant, when viewed from above, has the x branches distributed relatively evenly around the stem and around the first splitting, as shown in FIG. 3, i.e. preferably not all of the x branches are on one side of the plant, especially in a 4, 5, 6, 7 or 8-branch plant, so that the weight of the branches (which may be supported vertically by attaching a string or rope for support) is not all on one side of the plant.

In one embodiment one or more or all of the 3, 4, 5, 6, 7 or 8 are supported vertically by at least one string, twine or rope. This may be done by having overhead horizontal means of attaching the twine, string or rope.

As mentioned further above, the un-pruned splitting(s) following the first splitting can be at any node following the first splitting. However, in one aspect the un-pruned splitting(s) are the splitting(s) most near to the first splitting, i.e. preferably at node 1, 2 or 3 (where the first splitting is node ‘0’ and all further nodes/splittings are counted upwards from node ‘0’ as shown in FIGS. 1 and 2). In this way, the plant retains higher stability. However, when pruning one should aim to retain strong shoots and branches and therefore remove weaker heads or shoots (or branches). The weakness of a head or shoot is judged visually. Typically a head or shoot is judged to be weaker as compared to another shoot if it is e.g. thinner, shorter, has abnormal structure and/or is more affected by any negative force such as, but not limited to, mechanical stress, temperature stress, radiation stress and/or stress due to a pathogen.

Preferably, removing of supernumerary heads or shoots is done as soon as the head or shoot is big enough to be removed easily. The earlier the head or shoot is removed the less energy/effort are lost for unnecessary parts.

Preferably, pruning is performed every 3, 4, 5, 6, 7 or 8 days, preferably every 3, 4 or 5 days, more preferably every 5 days, after providing said first parent plant as defined herein to result in a plant comprising the specific number of x branches. Pruning may be continued as long as necessary to keep the specific number of x branches.

Thus, a plant comprising 3, 4, 5, 6, 7 or 8 branches is generated and maintained by pruning. The resulting Capsicum plant and the respective pruning system is denominated herein as a 3, 4, 5, 6, 7 or 8 branch architecture. Especially good results were achieved in the method of the invention with a 3, 4 or 5 branch architecture. Therefore in one aspect the method of the invention generates a plant comprising a 3, 4 or 5 branch architecture.

In one aspect the 3-branch architecture is formed and maintained by pruning every 3, 4, 5, 6, 7 or 8 days, after the first splitting has formed and/or after 3 branches have been formed; the 4 branch architecture is formed and maintained by pruning every 3, 4, 5, 6, 7 or 8 days the first splitting has formed and/or after 4 branches have been formed; the 5-branch architecture is formed and maintained by pruning every 3, 4, 5, 6, 7 or 8 days the first splitting has formed and/or after 5 branches have been formed; the 6 branch architecture is formed and maintained by pruning every 3, 4, 5, 6, 7 or 8 days the first splitting has formed and/or after 6 branches have been formed; the 7-branch architecture is formed and maintained by pruning every 3, 4, 5, 6, 7 or 8 days after the first splitting has formed and/or after 7 branches have been formed; the 8 branch architecture is formed and maintained by pruning every 3, 4, 5, 6, 7 or 8 days after the first splitting has formed and/or after 8 branches have been formed.

Also provided is a plurality of pruned Capsicum plants, e.g. a plurality of pruned inbred lines of a cultivated pepper, produced by any of the herein described methods, is provided.

In one embodiment of the invention also a plurality of pruned Capsicum plants of a specific line (preferably an inbred line, especially an inbred parent line of a cultivated pepper variety) is provided, pruned according to any of the methods described herein, wherein all the pruned plants have a specific branch architecture selected from 3-branch, 4-branch, 5-branch, 6-branch, 7-branch or 8-branch. Thus in one aspect all the pruned plants have the same specific branch architecture selected from 3-branch, 4-branch, 5-branch, 6-branch, 7-branch or 8-branch. All the plants are of an inbred line of the same genotype, preferably an inbred parent line of cultivated pepper (e.g. an inbred parent line of a cultivated pepper variety), preferably of sweet pepper.

In one aspect the plurality of pruned Capsicum plants comprise at least one branch of each plant which is supported with at least one vertical twine, string or rope. However, in another aspect several of the specific branches or even all of the specific branches of each plant are supported with at least one vertical twine, string or rope.

The plurality of pruned Capsicum plants are capable of producing (and do produce) an average mature seed yield per plant that is at least 10% higher than the average mature seed yield per plant produced by a plurality of pruned Capsicum plants of an inbred parent line of cultivated pepper having been pruned to the conventional 2-branch architecture.

In one aspect the pruned plants are male sterile or comprise emasculated flowers. In a further aspect the emasculated flowers have been pollinated with pollen of a different Capsicum plant, e.g. pollen of an inbred line, especially an inbred male parent line of a cultivated pepper variety.

The pruning architecture of the present invention results in a reduced pollination period as compared to the conventional 2-branch architecture. A shorter pollination period is economically beneficial because of a reduced cultivation- and seed production-time. A further beneficial aspect of the method of the invention is labour saving. Labour is saved in several ways, e.g., (i) maintenance of more branches in the method of the invention implies less pruning labour, (ii) in the method of the invention flowers develop closer together and/or on the same height, resulting in less labour required for hand pollination, and (iii) in the method of the invention fruit develops more simultaneous, decreasing fruit collection labour.

Emasculating the flowers growing on the first parent plant in step c) is performed in order to avoid self-pollination. Emasculation is preferably done by hand, e.g. using tweezers. In an alternative of the method, the plant of step a) comprises genetic or cytoplasmic male sterility, so that emasculation of the flowers is not necessary. In this embodiment, step c) can be omitted from the method. An advantage is that then natural pollination can be used in step d), although hand pollination is also an option in this case.

Allowing pollination of the emasculated, or male sterile, flowers with the pollen of a second parent plant in the method of the invention, in step d), is understood herein as to encompass both natural pollination, e.g. in case of male sterile flowers, and hand pollination, e.g. in case of emasculated flowers. Hand pollination may also be referred to as mechanical pollination. Natural pollination may be, but is not limited to, pollination via insects such as bumblebees and wind.

Preferably, in a method of the invention step d) pollination is done by hand. Typically, in hand pollination, pollen collected from the second parent plant is delivered to the stigma of flowers of the first parent plant. Obtaining pollen from the second parent plant can be done as known by the skilled person, e.g., by picking flowers or anthers of the second plant and rubbing these onto the stigma of the first parent plant. Alternatively, pollen may be separated from the anthers, e.g. by sieving the anther and collecting the pollen in a glass tube. Pollination may be performed at a time after emasculation of the first parent plant when the stigma is most susceptible for pollination, e.g. about 2 days after emasculation, depending on the female Capsicum parent used.

The method may be applied in any region suitable for pepper seed production, which are generally warm climates, but the method can also be applied in cooler climates. Thus, in one aspect the method of the invention is applied in any region suitable for pepper seed production. In another aspect the method of the invention is carried out in countries with a cool environment such as, but not limited to, North America (e.g. Canada, USA) and northern Europe (e.g. The Netherlands, Germany, Belgium, Scandinavia, UK, etc.). The methods of the invention may be carried out in known environments for pepper seed production, e.g. in a nethouse, tunnel, glasshouse, or even in the field. A greenhouse is preferably understood to be glasshouse as known in the art. Also useable is a plastic greenhouse (e.g., a double layer polyethylene singe type greenhouse) a climate chamber or a net house (e.g., referred is to Satpute et al., 2013; Maboko et al., 2012), a plastic tunnel (e.g., referred is to Maniutiu et al., 2010), as known in the art. The method of the invention may be performed in any time period suitable for pepper seed production, such as e.g. spring cultivation. Typically, pepper plants in spring cultivation in the Netherlands are seeded early in the year in the nursery, and transplanted into the greenhouse or net-house or tunnel. Pollination is performed in spring, after which and the fruits and seeds are allowed to mature up to early summer, when the fruits are harvested. In one aspect seeds are allowed to mature for at least 50, 55, 57, 58, 59, 60, 61, 62, 63, 64, or 65 days after pollination.

In step f) the fruits are harvested, e.g. picked by hand, and the seeds are collected from the harvested fruits.

In principle fruits can be harvested and opened to collect seeds as soon as the seeds are formed, i.e. even when seeds are not fully matured, so for example immature or semi-mature fruits can be harvested, but it is preferred that the fruits are mature or even over-mature, as then a higher percentage of seeds will be mature. Thus in one aspect fruits are harvested in step f) when the majority of seeds are mature, i.e. to maximize mature seed yield. Thus in one aspect fruits are harvested at a stage when they are not marketable anymore for human consumption as they would be considered as having a too short shelf-life or being over-mature (softening of the fruit flesh). In one aspect fruits are harvested at or after about 50 days, 55 days, 60 days, or 65 days after pollination. The harvested seeds can be treated by one or more methods as described previously. Alternatively, mature seeds can be separated from the other seeds, so that a high percentage of seeds is mature (e.g. at least 80%, 90%, 95%, 97%, 98%, 99% or 100%), which can then be treated by one or more treatments methods and eventually packaged for sale. The seeds are preferably F1 hybrid pepper seeds of a cultivated pepper variety, e.g. of a sweet pepper variety.

Mature seeds can be separated from less mature seeds and/or immature white seeds using different methods known to the skilled person. In one aspect the harvested seeds are placed in water, as the mature seeds are heavier than the less mature and white immature seeds and will sink (while less mature and immature seeds float). This way the mature seeds can easily be collected.

However, alternative methods exist for separating mature seeds from less mature seeds. For example Burcu Begüm Kenanoglu, Ibrahim Demir, and Henk Jalink describe in HortScience 48:965-968 the use of chlorophyll fluorescence sorting to separate less mature seeds from mature seeds. They found that seeds harvested from half-mature and mature fruits and then sorted using chlorophyll fluorescence sorting gave higher quality seeds in terms of germination, emergence and vigor.

Thus, in one aspect the fruits harvested in step f) may be half-mature, mature or over-mature.

The first and/or second parent plants of the method of the invention may be grown in soil (full soil or in pots), perlite bags, on an aerated nutrient solution, or on rockwool, or hydroponically (e.g., on sawdust or rockwool, while irrigated with a nutrient solution as known in the art) (e.g., referred is to Jovicich et al., 1999; Nielsen and Veirskov, 1988; Maboko and Ciloane, 2012. In one embodiment the first parent plants of the method of the invention are grown on rockwool. In another embodiment the first parent plants of the method of the invention are grown in the full soil, e.g. in the field.

Plants may be grown under any conditions suitable for growing Capsicum, especially cultivated Capsicum plants. In one aspect the plants are grown in controlled environment conditions, such as a greenhouse. Thus in one aspect the first parent plants are grown under standard greenhouse conditions for Capsicum growth, e.g. on rockwool, with e.g. an average day temperature of about 23° C. (±0.5° C.) and an average night temperature of about 19° C. (±1.0° C.) and a relative humidity of 60-85%.

The climate may be a controlled climate, i.e. a climate controlled in the sense that temperature, humidity and radiation are tightly regulated by means known in the art.

Plants can be grown at various densities. So for example in the method of the invention the first parent plant has a planting density of 1.0 1.5 2.0, 2.1, 2.2, 2.3, 2.4, 2.5, 2.6, 2.7, 2.8, 2.9, 3.0, 3.1, 3.2, 3.3, 3.4 or 3.5, 4.0, 4.5 or 5.0 plants/m2, more preferably 2.4 to 3.2 plants/m2, most preferably 2.6 to 3.0 plants/m2.

Depending on the predetermined number of branches, this will lead to a certain branching density per square meter. So for example when 4-branch plants are grown at a density of 2.6 plants/m2, the resulting branching density is 4×2.6=10.4 branches per m2.

In one embodiment of the invention the first parent plant is grown at a branching density of about 7, 8, 9, 10, 11, 12, 13, 14, 15, 16, 17, 18, 19, 20, 21, 22, 23, 24, 25, 26, 27, 28 or 30 branches/m2 and the first parent plant is pruned to a specific number of branches selected from 3, 4, 5, 6, 7 or 8 branches. In one aspect the first parent plant is grown at a branching density of about 13, 14, 15, 16, 17, 18, 19, 20 or 21 branches/m2 and the first parent plant is pruned to a specific number of branches selected from 3, 4, 5, 6, 7 or 8 branches. So for a 4-branch plant to be grown at a planting density of about 13 branches per m2, plants should be grown at a density of 3.1 to 3.35 plants per m2 and pruned to a 4-branch architecture. A branching density of ‘about 7’, means a branching density of 6.5, 6.6, 6.7, 6,8, 6.9, 7.0, 7.1, 7.2, 7.3, or 7.4; a branching density of ‘about 8’, means a branching density of 7.5, 7.6, 7.7, 7,8, 7.9, 8.0, 8.1, 8.2, 8.3, or 8.4; etc.

In one aspect in the method of the invention the first parent plant has a 3 branch architecture and a branching density of about 8 or about 9 branches/m². In a different aspect in the method of the invention the first parent plant has a 4-branch architecture and a branching density of about 10, about 11 or about 12 branches/m². In a different aspect in the method of the invention the first parent plant has a 5-branch architecture and a branching density of about 13, about 14 or about 15 branches/m²In a further aspect in the method of the invention the first parent plant has a 6-branch architecture and a branching density of about 15, about 16, about 17 or about 18 branches/m². In yet a further aspect in the method of the invention the first parent plant has a 7-branch architecture and a branching density of about 18, about 19, about 20 or about 21 branches/m². In still a different aspect in the method of the invention the first parent plant has an 8-branch architecture and a branching density of about 20, about 21, about 22, about 23 or about 24 branches/m².

The method of the invention results in an increase in total average seed yield per plant as defined herein of at least 10%, 20%, 30%, 40%, 50%, 60%, 70%, 80%, 90%, 100%, 110%, 120%, 130%, 140% or even 150% as compared to the total average seed yield per plant when the same method is applied under the same conditions with the exception that first parent plant is pruned to the conventional 2 branch architecture in step b). Thus the total average seed yield of seeds harvested in step f) of the method is significantly increased compared to the seed yield of the control plants grown under the same conditions but pruned to a 2-branch architecture.

In one aspect the method of the invention results in an increase in total average mature seed yield per plant as defined herein of at least 10%, 20%, 30%, 40%, 50%, 60%, 70%, 80%, 90%, 100%, 110%, 120%, 130%, 140% or even 150 as compared to the total average mature seed yield per plant when the same method is applied under the same conditions with the exception that first parent plant is pruned to the conventional 2 branch architecture in step b). Thus the total average seed yield of mature seeds harvested in step f) and optionally separated from less mature or immature seeds in step g) of the method is significantly increased compared to the mature seed yield of the control plants grown under the same conditions but pruned to a 2-branch architecture.

The method of the invention in one aspect also results in an increase in total average fruit yield per plant as defined herein of at least 10%, 20%, 30%, 40%, 50%, as compared to the total average fruit yield per plant when the same method is applied under the same conditions with the exception that first parent plant is pruned to the conventional 2 branch architecture in step b).

Provided is also s method for pruning a Capsicum plant, comprising the steps:
a) providing a plurality of plants which comprises a stem, a first splitting and two branches; and
b) pruning the plants to a specific number of branches selected from 3, 4, 5, 6, 7 or 8, and optionally supporting at least one branch with at least one vertical twine, string or rope.

In one aspect the pruning methods above may further comprising the steps:
c) emasculating flowers produced on said plants; and optionally
d) allowing pollination of said flowers with the pollen of a second inbred line; and optionally
e) allowing fruits to grow; and optionally
f) harvesting said fruits and collecting seeds from the fruits of e).

In another aspect a method for pruning an inbred line of a Capsicum plant is provided comprising the steps:
a) providing a plurality of plants of the inbred line, wherein each plant comprises a stem, a first splitting and two branches; and
b) pruning the plants to a specific number of branches selected from 3, 4, 5, 6, 7 or 8, and optionally supporting at least one branch with at least one vertical twine, string or rope.

The pruning methods may further comprising the steps:
c) emasculating flowers produced on said plants; and optionally
d) allowing pollination of said flowers with the pollen of a second inbred line; and optionally
e) allowing fruits to grow; and optionally,
f) harvesting said fruits and collecting seeds from the fruits of e).

The pruning methods are essentially as described herein further above in relation to the methods for producing seeds of Capsicum, i.e. whereby plants are pruned throughout their growth to result in adult plants which have a specific number of main branches. Thus, the embodiments which are described in relation to the seed production methods of the invention equally apply to the pruning methods of the invention.

The plants may be pruned every 3, 4, 5, 6, 7 or 8 days after providing the plant under step a), preferably every 5 days.

In one aspect the weakest or inward facing head or shoot is removed at nodes that are pruned. The method thus results in adult plants where 3, 4, 5, 6, 7 or 8 branches are left after pruning.

In one aspect the plurality of plants which are pruned are plants of an inbred parent line of cultivated pepper of the species C. annuum, preferably the plurality of plants are of the same inbred parent line. In one aspect the inbred parent line is a parent of an F1 hybrid pepper variety, especially a variety of sweet pepper.

In a further aspect the plants of step a) are grown at a density of 10, 11, 12, 13, 14, 15, 16, 17, 18, 19, 20 or 21 branches per square meter.

As described before, at least one branch or more, e.g. at least 2, 3, 4, 5, 6, 7 or all branches, may be supported with at least one vertical support, such as a twine, string or rope.

The plants of step a) are in one aspect inbred parent lines of a pepper type selected from the group consisting of: Bell (blocky), Lamuyo type, Dulce Italiano, Dulce California, Dulce Hungario, snack type pepper, conical, capia, sweet charleston, dolma, cherry, jalapeno, shakira, pencil or hot Charleston, Sivri, Hungarian wax, Kapya/Florinis, banana, Fresno, Serrano, Ancho, Anaheim, Pasilla, Santa Fe, Scotch bonnet, and Habanero.

In one embodiment of the invention also a plurality of pruned Capsicum plants of a specific line (preferably an inbred line, especially an inbred parent line of a cultivated pepper variety, especially a variety of sweet pepper) is provided, pruned according to any of the methods described herein, wherein all the pruned plants have a specific branch architecture selected from 3-branch, 4-branch, 5-branch, 6-branch, 7-branch or 8-branch.

In one aspect the pruned plants are male sterile or comprise emasculated flowers. In a further aspect the emasculated flowers have been pollinated with pollen of a different Capsicum plant, e.g. pollen of an inbred line, especially an inbred male parent line of a cultivated pepper variety. In one aspect the inbred male parent line is a parent of an F1 hybrid pepper variety, especially a variety of sweet pepper.

### Definitions

The indefinite article “a” or “an” does not exclude the possibility that more than one of the element is present, unless the context clearly requires that there be one and only one of the elements. The indefinite article “a” or “an” thus usually means “at least one”.

As used herein, the term “plant” preferably includes the whole plant, including different developmental stages, such as seedlings, immature and mature, etc. When “seeds of a plant” are referred to, these either refer to seeds from which the plant can be grown or to seeds produced on the plant, after fertilization. Which meaning is used is evident from the context.

“Plant variety” is a group of plants within the same botanical taxon of the lowest grade known, which (irrespective of whether the conditions for the recognition of plant breeder's rights are fulfilled or not) can be defined on the basis of the expression of characteristics that result from a certain genotype or a combination of genotypes, can be distinguished from any other group of plants by the expression of at least one of those characteristics, and can be regarded as an entity, because it can be multiplied without any change. Therefore, the term “plant variety” cannot be used to denote a group of plants, even if they are of the same kind, if they are all characterized by the presence of one or two loci or genes (or phenotypic characteristics due to these specific loci or genes), but which can otherwise differ from one another enormously as regards the other loci or genes.

“F1, F2, F3, etc.” refers to the consecutive related generations following a cross between two parent plants or parent lines. The plants grown from the seeds produced by crossing two plants or lines is called the F1 generation. Selfing the F1 plants results in the F2 generation, etc.

“F1 hybrid” plant (or F1 hybrid seed) is the generation obtained from crossing two inbred parent lines. Thus, F1 hybrid seeds are seeds from which F1 hybrid plants grow. F1 hybrids are more vigorous and higher yielding, due to heterosis. Inbred lines are essentially homozygous at most loci in the genome.

A “stem” or “plant stem” is the stalk of the plant and the major vertical shoot conducting water, minerals, and food to other parts of the plant. It is the main support of the plant.

A “head” is a growing point produced at the nodes of the shoot or branch.

A “shoot” or “branch” is understood herein as the newly grown aerial portion of the plant that grows out from the head. Generally when it is still young it will be referred to as a shoot and when it is more developed it will be referred to as a branch.

An “inward facing head, shoot or branch” is to be understood herein as a shoot that is growing in the direction of the axis of the plant, wherein the axis is to be understood as the line extrapolated from the stem of the plant.

“Node” is referred herein as the part of a plant where two growing points (heads) develop and in pepper the nodes are also the points where the leaves and flowers develop. A node is also denominated herein as “splitting”. “Internode” refers to the portion of a plant between any two adjacent nodes.

“First splitting” refers to the node where the developing pepper plant for the first time forms two growing points (at the end of the stem). In FIGS. 1 and 2 the first splitting is indicates as node 0 (zero).

“Pruning” is a technique used in a.o. horticulture, which involves the selective removal of plant parts. Plant parts that are commonly pruned encompass stems, branches, vines, leaf buds, flower buds, developing leafs, developing flowers and roots. The skilled person is aware of many methods and schemes for pruning. Various tools can be used by the skilled person for pruning, such as pruning shears, also known as hand pruners, scissors, scalpels or simply hands or finger tips.

A “pruning architecture” is to be understood herein as the result of a pruning method, preferably a pruning method as defined in step b) of the method of the invention. A “two-stem pruning architecture” or “V” trellis system consists of forming a plant with two main branches by removing one of the two heads or shoots that develop at each node. A pepper plant having a 3-branch architecture is to be understood herein as a pepper plant pruned to have 3 main branches, pepper plant having a 4 branch architecture is to be understood herein as a pepper plant pruned to have 4 main branches, and so on.

“Domesticated pepper” refers to the species Capsicum annuum L., Capsicum chinense Jacq., Capsicum frutescens L., Capsicum baccatum L., and Capsicum pubescens Ruiz & Pay.

The term “cultivated pepper” refers to breeding lines and varieties of domesticated pepper, which is cultivated by humans in the field or in protected environments (e.g. greenhouse or tunnels or net-houses) for fruit production. Cultivated pepper plants have good agronomic properties compared to wild accessions such as high yielding, bigger fruit size, higher fertility, higher uniformity of plants and/or fruits, etc. Examples of cultivars include cultivated varieties that belong to the species Capsicum annuum, Capsicum chinense, Capsicum frutescens, Capsicum baccatum and Capsicum pubescens. In a preferred aspect the term “cultivated pepper” refers herein to Capsicum annuum, which is the most cultivated species, including many sweet pepper varieties and hot pepper varieties.

“Sweet Pepper” refers herein to Capsicum fruits and the plants they grow on that are not perceived as pungent by humans, because they e.g. have less than approximately 16 heat units on the Scoville scale, e.g. 0 to 10 heat units. A sweet pepper variety is a variety which produces sweet pepper fruits, such as Bell pepper or “paprika”. See e.g. the world wide web at de.wikipedia.org/wiki/Scoville-Skala.

“Hot Pepper” refers herein to Capsicum fruits and the plants they grow that are perceived as pungent or hot by humans, because they e.g. have over 16 heat units on the Scoville scale, such as 100 or more scoville heat units, and contain discernable capsaicinoids. See e.g. the world wide web at de.wikipedia.org/wiki/Scoville-Skala.

The “Scoville scale”, “Scoville Heat Unit (SHU)” or Scoville Units is measurement of the pungency (spicy heat) of peppers as reported in Scoville heat units (SHU), a function of capsaicin concentration. The scale is named after its creator, American pharmacist Wilbur Scoville. The Scoville scale can be measured empirically, dependent on the capsaicin sensitivity of testers (i.e. by tasting the pepper or tasting diluted pepper samples until heat no longer can be detected by the tasters. Alternatively, high-performance liquid chromatography (HPLC) can used to determine SHU. In this procedure, fruits are dried and then ground, next, the capsaicinoids, compounds responsible for heat or pungency are extracted, and the extract is injected into the HPLC for analysis.

“Capsaicinoids” refers herein to the family of compounds that are mainly responsible for the pungent taste of pepper. It encompasses six compounds capsaicin, dihydrocapsaicin, nordihydrocapsaicin, homodihydrocapsaicin, homocapsaicin and nonivamide. Capsaicin is the most common and most pungent capsaicinoid in peppers, followed by dihydrocapsaicin. The other capsaicinoids have limited contribution to the pungency of peppers.

“Pungency” or heat refers herein to the spicy “hot” taste caused mainly by capsaicinoids.

“Sowing” refers to the process of placing the seed in an appropriate medium for germination, so that it is able to grow into a plant. The medium can e.g. encompass soil, various types of rockwool, (liquid) culture media, etc.

A “seed treatment formulation” is understood herein to be a formulation comprising at least one active ingredient such as, but not limited to, a pesticide, a seed nutrient or a seedling disease treatment agent. Preferably, said at least one active ingredient is a pesticide such as an insecticide, a fungicide, an antimicrobial, an acaricide, a nematicide, a fungicide and/or a combination hereof. Seed treatment formulations are understood to possibly comprise a combination of different active ingredients. Such formulation is preferably added to the seed in the form of a film coating, which is a uniform, dust-free, water permeable film, evenly covering the surface of all individual seeds (e.g., referred is to Halmer, 2000) The seed treatment formulation can be added directly on the seed or in a pelleting mixture as define herein. Besides active ingredient(s), the seed treatment formulation generally also contains other ingredients such as water, glue (typically a polymer), filler materials, pigments and certain additives to improve particular properties of the coating. A variety of techniques and machines exist to apply film coatings, and many of these can also be used or adapted for seed pelleting. Manufacturers of seed treatment machines are, for example, Gustafson Equipment, Satec and SUET. Techniques and machines vary in the method of applying the seed treatment mixture to the seed and the blending process (Jeffs, K. A. and Tuppen, R J. 1986. Applications of pesticides to seeds. Part 1: Requirements for efficient treatment of seeds. In: Seed treatment. Ed: Jeffs, K. A.). The mixture, for example, can be added by means of a spinning disc atomizer or spreading brushes. The seeds and the mixture can be blended by means of an auger, in a drum, or in rotating troughs. If the amount of film coating mixture added is low, and can be absorbed by the seed itself with only a slight (typically less than 1%) increase in seed moisture content, no additional drying step is necessary. This principle is called self-drying (Black et al., 2006. The encyclopedia of seeds. Science, technology and uses). Otherwise, a drying powder (such as talc) could be added, or an additional drying step is necessary. This step could be integrated in the equipment for film coating, such as in the SUET rotary seed treater with integrated fluid bed dryers. Some SATEC batch coaters are equipped to be connected with drying air also.

“Seed pelleting” is a technique that is primarily intended to change the natural shape and size of the raw seed. These techniques in general comprise adding a pelleting mixture in order to change seed size and/or shape for creating round or rounded seed shapes which are easily sown with modern sowing machines (e.g., referred is to Halmer, 2000).

A “pelleting mixture” contains at least glue and filler material. The latter could be, for example, clay, mica, chalk or cellulose. In addition, certain additives can be included to improve particular properties of the pellet. A seed treatment formulation as defined herein can be added directly into the pelleting mixture. It is also encompassed that a seed treatment formulation is added on the outside of the pellet, for instance between two layers of pelleted seed, or directly on the seed before the pelleting material is added. Also more than 1 film coating layer of seed treatment formulation can be incorporated in a single pellet.

“Pollination” is the process by which pollen is transferred to the stigma of the flower, thereby enabling fertilization and reproduction.

“Emasculation” is understood herein as the removal of the anthers to prevent self-pollination.

A “pruned splitting” is a splitting (or node) from which one of the two heads (or growing points) at the splitting (or node) is removed.

An “un-pruned splitting” is a splitting (or node) at which both heads (or growing-points) formed at that splitting are kept.

“Seed yield per plant” or “average seed yield per plant” or “average total seed yield per plant” is understood herein to be the total average number of seeds (immature, less mature and/or mature) produced per plant when a plurality of plants are grown and pruned in the same way according to the method of the invention. In one aspect the average total seed yield per plant can be established by determining the average total number of fruits produced per plant (excluding parthenocarpic fruits), and multiplying this number with the average number of seeds per fruit. The average seed number per fruit can be determined as soon as the seeds have formed, so e.g. fruits can be harvested when the fruits are immature, half mature, mature or overmature, as the seed number doesn't change once the seeds have formed. The average number of seeds per fruit can be determined e.g. by counting all of the seeds of all fruits produced by a plant (excluding parthenocarpic fruits), and by doing this for a number of plants e.g. at least three, four or more plants grown and pruned in the same way, to calculate the average seed number per fruit. The average total number of fruits produced per plant can be determined by growing and pruning a plurality of plants in the same way (e.g. at least 3, 4, 5, 6, 7, 8, 9, 10, 12, 15, 18, 20 or more) and by counting the fruits (excluding parthenocarpic fruits) and calculating the average number of fruits. It is understood that this should not be done too early, as otherwise fruits may be counted which may later be aborted. “Significantly more” refers herein to a statistically significant increase compared to the control. So for example a plurality of pruned plants (pruned according to the method of the invention, selected from a 3, 4, 5, 6, 7, or 8 branch architecture) which produce significantly more average numbers of fruits and/or significantly more total average seed yield per plant than the control plants (pruned to a 2-branch architecture), refers to a statistically significantly increase at a significance level of 1% (p<0.01 using ANOVA) or 5% (p<0.05 using ANOVA). It is understood that a plurality of plants, e.g. at least about 3, 5, 8, 10, 15, 20 or more plants are grown under the same conditions and pruned in the same way (to a 3, 4, 5, 6, 7, or 8 branch architecture) to obtain averages. In addition a plurality of control plants, e.g. at least about 3, 5, 8, 10, 15, 20 or more control plants are grown under the same conditions and pruned to a 2-branch architecture to obtain averages for the control.

“Average” or “mean” refers herein to the arithmetic mean and both terms are used interchangeably. The term “average” or “mean” thus refers to the arithmetic mean of several measurements. The skilled person understands that arithmetic means of at least 3, 5, 8, 10, 15, 20, 30, 40, 50 or more plants and/or plant parts are measured, preferably in randomized experimental designs with several replicates and suitable control plants grown under the same conditions in the same experiment. “Statistically significant” or “statistically significantly” different or “significantly” different refers to a characteristic of a plant line that, when compared to a suitable control (e.g. herein the plant line having a 2-branch architecture) show a statistically significant difference in that characteristic (e.g. the p-value is less than 0.01 or less than 0.05 using ANOVA) from the (mean of the) control.

“Harvest” is understood herein to be the collection of pepper fruits and/or seeds developed in the fruits.

“Mature seeds” refers to seeds that have fully matured in the fruits.

“Less mature seeds” and “immature seeds” refers to seeds that are not yet fully mature in their development and have not reached their final mature seed color but are e.g. white (immature) or have various degrees of pigment but to a lesser extent than the fully mature seeds.

A “plant line” or “breeding line” refers to a plant and its progeny. As used herein, the term “inbred line” refers to a plant line which has been repeatedly selfed and is nearly homozygous. Thus, an “inbred line” or “parent line” refers to a plant which has undergone several generations (e.g. at least 4, 5, 6, 7 or more) of inbreeding, resulting in a plant line with a high uniformity.

A “female plant” is to be understood herein as a plant that is pollinated by a male parent.

A “male plant” is to be understood herein as a plant of which the pollen is used to pollinate a female plant.

A “double haploid” (or DH) plant is a plant generated from a haploid cell by doubling the chromosomes of that cell and regenerating a plant therefrom.

“Selfing” refers to self-pollination of a plant, i.e., the transfer of pollen from the anther to the stigma of the same plant.

“Crossing” or cross-pollination refers to the mating of two parent plants, e.g. pollinating a female parent line with pollen of a male parent line.

### REFERENCE LIST

Marcelis et al. (2004). Flower and fruit abortion in sweet pepper in relation to source and sink strength. Journal of Experimental Botany, Vol. 55, No. 406, pp. 2261-2268.

Jovicich et al., (2004) Fruit yield and Quality of Greenhouse-grown Bell pepper as influenced by density, Container, and Trellis system. Research Reports HortTechnology Vol 14 (4) p507-513.

Jovicich, Cantliffe and Hochmuth (1999) Plant density and shoot pruning on yield and quality of a summer greenhouse sweet pepper crop in Northcentral Florida. http://hos.ufl. edu/protectedag/pdf/pepperpruning-elio.pdf

Maniutiu, Sima, Apahidean, Apahidean and Ficior (2010) The Influence of Plant Density and Shoot Pruning on Yield of Bell Pepper Cultivated in Plastic Tunnel. Bulletin UASVM Horticulture, 67(1), p259-263.

Nielsen and Veierskov (1988). Distribution of Dry Matter in Sweet Pepper Plants (Capsicum annuum L.) during the Juvenile and Generative Growth Phases. Scientia Horticulturae, (35) p179-187.

Maboko, Du Plooy and Chiloane (2012) Effect of plant population, stem and flower pruning on hydroponically grown sweet pepper in a shadenet structure Agricultural. African Journal of Agricultural Research Vol. 7(11), pp. 1742-1748.

Satpute, Bharad, and Korde (2013) Effect of spacing and plant architecture on yield and economics of Capsicum under net house conditions. HortFlora Research Spectrum 2(2) p150-152.

Halmer (2000) Commercial seed treatment technology. In: Seed technology and its biological basis. Eds: Black, M. And Bewley, J. D.

### LEGENDS OF THE FIGURES

FIG. 1: Schematic drawing of a plant pruned to a 3-branch architecture. Numbers indicate nodes. The vertical line with numbers 1-7 illustrates the stem. The first splitting is indicated by ‘0’, from which point upwards the nodes are numbered again. The three branches are illustrated by lines having nodes 0 to 5, of which node 1 on the right side branch has not been pruned.

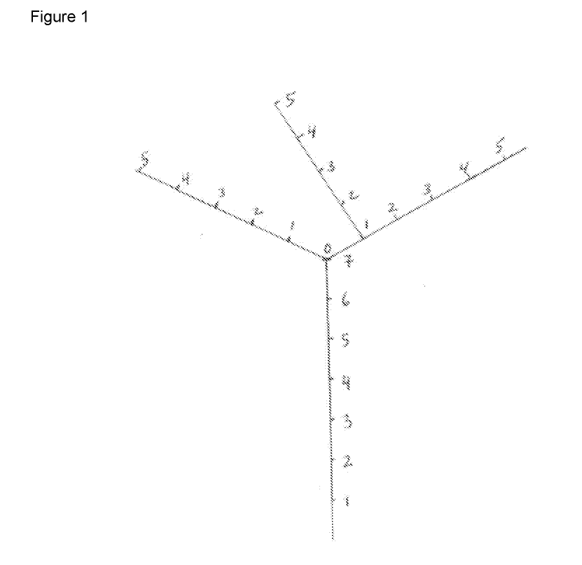

FIG. 2: Schematic drawing of a plant pruned to a 4-branch architecture. Numbers indicate nodes. The vertical line with numbers 1-7 illustrates the stem. The first splitting is indicated by ‘0’, from which point upwards the nodes are numbered again. The four branches are illustrated by lines having nodes 0 to 5, of which node 1 on the right side branch and on the left side branch has not been pruned.

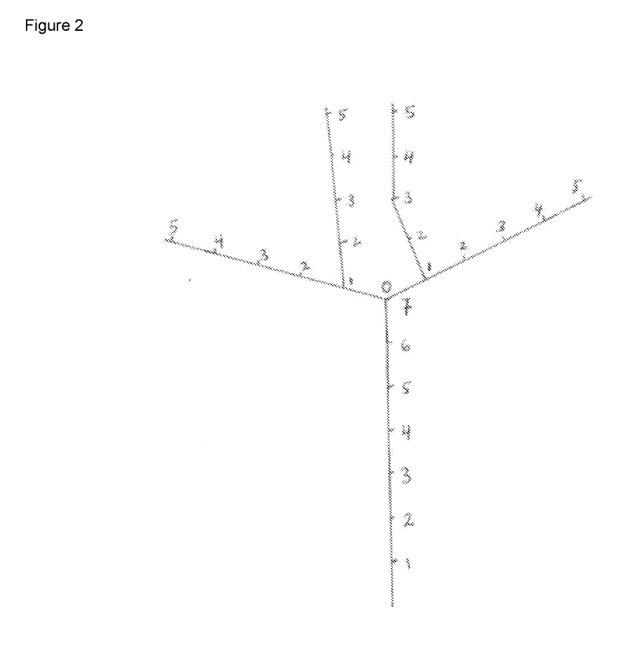

FIG. 3: Schematic drawing of a plant pruned to a 4-branch architecture from above, with the first splitting indicated by ‘0’.

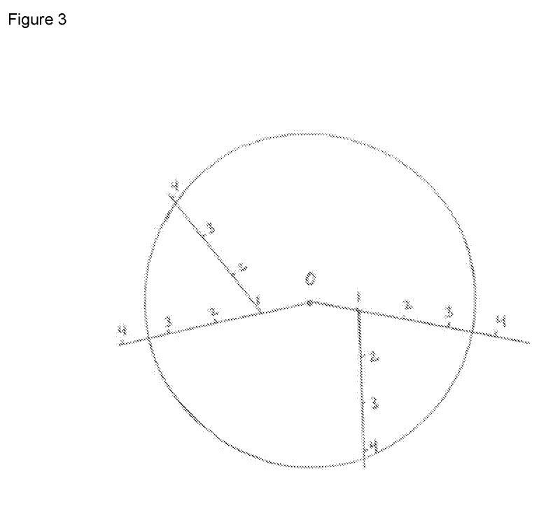

### EXAMPLE

### Materials and Methods

### Layout

Different pruning methods were tested in the spring cultivation season in a Dutch greenhouse. Plants were raised in a nursery from January to mid/end of February, when they were transplanted into the greenhouse and pruned to a particular architecture during growth. In the greenhouse plants were grown on rockwool slaps. Plants were grown in a randomized block design.

Pollination was carried out in the beginning of April, maturation of fruits and seeds took place from end of April until mid-June. Due to time-constraint fruits were already harvested mid/end of May, although they would normally be harvested end of June or in July. However, seed numbers are not affected by an early harvest, only seed maturity is affected.

### Pepper Varieties

For the inbred female parents of four pepper varieties (K1=Carson F1, K9=Bailor F1, K11=NUN55119 and K13=Soprano F1), the effect of pruning architecture on hybrid seed yield was tested in a Dutch greenhouse in the spring cultivation season. The female parents were grown in the greenhouse and pruned to a 2-branch (control), 3-branch or 4-branch or 7-8 branch architecture. Pollination was done with the male inbred parent, to generate F1 hybrid seeds in the fruits produced on the female parent.

K1 is a medium sized blocky pepper variety of Capsicum annum, which normally produces 10-12 fruits per plant under good growing conditions. The pollination period in this trial was 21 days from 21st March (first emasculation) till 12^{th} April (last emasculation).

K9 is a lamuyo pepper variety of Capsicum annum with big fruit size, which produces between 5-6 fruits in one cultivation cycle. The pollination period in this trial was 19 days from 22^{nd} March (first emasculation) until 11^{th} April (last emasculation). The pollination period of 19 days was the shortest in the trial. Further the plants were also the shortest in comparison to the other three varieties.

K11 is a big sized pointy pepper variety of Capsicum annum which produces 12-15 fruits per plant under good growing conditions. The pollination period of this variety was the longest in this trial with one month from 20^{th} March till 20^{th} April.

K13 is big sized pointy pepper variety of Capsicum annum with an average fruit production of 12-18 fruits per plant. The pollination period of this variety was 27 days from 22^{th} March till 19^{th} April.

### Treatments and Determinants

For the female parent of K1 and K9, the 2, 3 and 4 branch architecture was tested for 21 plants per branch architecture at 3 plants/m² and for 18 plants per branch architecture at 2.6 plants/m². For K11 and K13, the 2, 3, 4 and 7-8 branch architecture was tested for 18 plants per architecture at 2.6 plants/m². The respective 21 and 18 plants were spread over the greenhouse in 3 repetitions (a, b and c) of each 7 (total of 21) or 6 (total of 18) plants, respectively. A combination of a plant variety, pruning architecture and plant density is denominated herein as a “treatment”. Per treatment, the average number of flowers, average number of fruits, average seed yield per fruit and average total seed yield per plant was determined (by multiplying the average number of fruits per treatment with the average number of seeds per fruit).

As plants can only maintain a certain amount of flowers to grow out into fruit (due to abortion) a surplus of flowers were pollinated with pollen of the male parent line. The total average number of fruits per plant (excluding parthenocarpic fruits, which are easily recognized since they are shorter and misformed) was used as a base to calculate the total average seed yield per plant. The total average seed yield per plant of a particular treatment was established by multiplying the average number of fruits of that particular plant with the average number of seeds per fruit of that particular treatment. The average number of seeds per fruit of a particular treatment was established by harvesting all fruits from three representative plants of that particular treatment, counting the total number of seed comprised in these fruits and dividing this total number by the number of harvested fruits from these three plants.

### Trial Scheme

The trial started on February 28^{th} with transplanting the female parent lines of K1 and K9 plants from the nursery into the greenhouse. The same was done for K11 and K13 on March 5^{th}. Pollination occurred around March 20^{th} to around Apr. 20, 2014 (see above for the details per variety). Fruit counts, forming the basis of the results presented in Table 1, were performed on May 14^{th}, for K1 and K9 and on May 28^{th}, for K11 and K13.

### Pruning

The plants of the trial have been pruned every 5 days in order to obtain and maintain the specific pruning architecture of the specific plant, i.e. a 2-branch architecture (control plant), or a 3 or 4 or 7-8 branch architecture as defined herein. Pruning was done throughout the trial, up to and including harvesting the fruits. The 2-branch architecture was reached and maintained by keeping the two shoots of the first splitting and removing one of the two heads (or growing points) of each new internode formed after the first splitting. The 3 and 4 branch architecture was reached by keeping the two shoots of the first splitting and not pruning one (for the 3-branch architecture) or two (for the 4 branch architecture) or 5-6 (for the 7-8 branch architecture) later nodes and removing all supernumerary heads or shoots. The supernumerary heads or shoots were removed as soon as the shoot was big enough to be removed easily. For each pruning architecture, the weakest and latest formed head or shoot of the node was removed. Mostly, the weakest head or shoot of a node is the inward facing head or shoot of that node. However, in case the outward facing head or shoot of a node was weaker as compared to the inward facing one, the weakest head or shoot was removed.

### Pollination

Pollination was performed by hand in order to produce hybrid seed. Female inbred lines were emasculated to prevent self-pollination and the male inbred line was used to collect the pollen used to pollinate the female line by hand. To collect the pollen, all flowers on the male line were picked and then the anthers were removed and collected. The anthers were sieved and the pollen was collected. Small glass tubes, a bit wider than the stigma of the pepper flower, were filled with the pollen. The tube was gently pushed against the stigma to pollinate the flowers (pollination). To check the right moment of pollination, one (mostly emasculated) flower was marked to assess the moment the flower opens as this would be the stage the emasculated flower would be naturally pollinated. The pollination process was mostly done two days after emasculation when the stigma is perfectly susceptible for pollination, in the morning from 9.00 to 12.00 AM.

### Statistical Analysis

Per treatment, the effect of plant density and branch architecture on average fruit number, average seed yield per fruit, and average seed yield per plant was established. All data collected was statistically tested with a Univariate test to investigate effects of and interactions between treatment variables. In case of a significant effect or interaction was found, a post hoc analysis was executed to investigate the effects or interaction in detail. The program used for the analyses was IBM SPSS 19. Effects and interactions with a p-value of less than 0.05 were indicated as significant.

### RESULTS

Table 1 shows the average amount of seeds per fruit, the average amount of fruits and the average amount of seeds per plant per treatment.

TABLE 1

| | Plant | | Average | Average | Average | |
|---|---|---|---|---|---|---|
| | density | Branch | amount of | amount of | amount of | % of |
| | (plants/ | architec- | fruits* per | seeds per | seeds per | con- |
| Variety | m2) | ture | plant | fruit | plant | trol |
| K1 | 3 | 2-branch | 5.4 | 55.4 | 301 | 100% |
| | | (control) | | | | |
| | | 3-branch | 6.0 | 95.0 | 565 | 187% |
| | | 4-branch | 6.9 | 103.2 | 708 | 235% |
| | | 7-8-branch | 7.4 | 74.4 | 553 | 183% |
| | 2.6 | 2-branch | 5.4 | 82.7 | 446 | 100% |
| | | (control) | | | | |
| | | 3-branch | 6.9 | 87.5 | 603 | 135% |
| | | 4-branch | 6.7 | 74.0 | 498 | 111% |
| | | 7-8-branch | 8.1 | 98.4 | 793 | 177% |
| K9 | 3 | 2-branch | 4.3 | 94 | 408 | 100% |
| | | (control) | | | | |
| | | 3-branch | 5.2 | 135 | 698 | 171% |
| | | 4-branch | 6.4 | 132 | 841 | 206% |
| | | 7-8-branch | 7.8 | 135 | 1058 | 259% |
| | 2.6 | 2-branch | 4.7 | 108 | 509 | 100% |
| | | (control) | | | | |
| | | 3-branch | 5.7 | 123 | 705 | 138% |
| | | 4-branch | 6.5 | 166 | 1081 | 212% |
| | | 7-8-branch | 7.3 | 121 | 884 | 173% |
| K11 | 2.6 | 2-branch | 7.6 | 207 | 1568 | 100% |
| | | (control) | | | | |
| | | 3-branch | 9.6 | 211 | 2032 | 129% |
| | | 4-branch | 9.9 | 209 | 2066 | 131% |
| | | 7-8-branch | 10.9 | 201 | 2184 | 139% |
| K13 | 2.6 | 2-branch | 9.0 | 254 | 2283 | 100% |
| | | (control) | | | | |
| | | 3-branch | 10.8 | 245 | 2649 | 116% |
| | | 4-branch | 12.4 | 223 | 2759 | 120% |
| | | 7-8-branch | 13.1 | 246 | 3228 | 141% |

*excluding parthenocarpic fruits

Below the results per variety are given in further detail.

### K1

No significant effect of plant density on fruit production was found (p=0.113). A significant increase in total average number of un-aborted and seed containing fruit was found per increased number of branches (p<0.013), except between 3 and 4 branches (p=0.264). No significant effect of plant architecture and/or plant density on amount of seed per fruit was found. The average amount of seed per plant increased significantly relative to the control per increasing number of branches in the pruning architecture, but there was no significant difference between the 3 and 4-branch architecture. No significant difference was found between different plant densities within pruning architectures.

### K9

A significant interaction between average fruit number and pruning architecture was found (p=0.00). No significant effect of plant architecture and/or plant density on average numbers of seeds per fruit was found. In general, a significant increase in the average number of seeds per plant was found with an increased number of branches. There was however no significance difference between the 4-branch and the 7-8 branch architecture. No effect of plant density on total seed yield was found.

### K11

A significant interaction between average fruit number and pruning architecture was found (p=0.00). The average number of fruits per plant increased significantly with an increase in branch number, for example the 3-branch architecture resulted in a significant increase in the average number of fruits per plant compared to the control (p<0.01). There was however no significant difference between the 3, 4 and 7-8 branch architecture regarding average number of fruits produced. Total average seed number per plant increased significantly in the 3-branch, 4-branch and 7-8 branch architecture as compared to control.

### K13

The 3-branch, 4-branch and 7-8 branch architecture resulted in a significant increase in average number of fruits compared to the control (p<0.01). There was no significant difference between the 4 and 7-8 branch architecture. No significant effect of branch number on average number of seeds per fruit was found. The average number of seeds per plant increased significant for the 3-branch, 4-branch and 7-8 branch architecture, but there was no significant difference between the 3-branch and 4-branch architecture.

### CONCLUSION

As can be seen from Table 1 above the average amount of F1 hybrid seeds produced per plant was significantly higher in a 3-branch, 4-branch and 7-8 branch architecture compared to the control. A further benefit of a pruning architecture of 3, 4 or 7-8 branches is that the pollination period was shorter, since the fruit were found to develop faster and more simultaneous. It was found that the pollination period can be shortened by at least 6 days without negatively influencing seed production (data not shown). Due to lower labour costs, a shorter pollination period will result in cost saving. In addition, it was found that an increase in the number of branches per m² had a positive effect on seed yield.

1. A method for pruning an inbred line of a Capsicum plant, comprising:
pruning a plurality of plants of the inbred line, wherein each plant comprises a stem, a first splitting and two branches,
to a specific number of branches selected from 3, 4, 5, 6, 7 or 8, and optionally supporting at least one branch with at least one vertical twine, string or rope.

2. The method of claim 1, further comprising:
emasculating flowers produced on said plants;
allowing pollination of said flowers with the pollen of a second inbred line;
allowing fruits to grow; and optionally,
harvesting said fruits and collecting seeds from the fruits.

3. A method for producing seeds of Capsicum, comprising:
pruning a first parent plant, wherein the plant is an inbred line,
to a specific number of branches selected from 3, 4, 5, 6, 7 or 8;
emasculating flowers produced on said plant;
allowing pollination of said flowers with the pollen of a second parent plant, wherein the plant is an inbred line;
allowing fruits to grow; and optionally,
harvesting said fruits and collecting seeds from the fruits.

4. The method of claim 2, further comprising separating mature seeds from the less mature and/or immature seeds.

5. The method according to claim 3, wherein average mature seed yield per plant is increased by at least 10% compared to the average mature seed yield per plant when the same method is applied under the same conditions with the exception that the first plant is pruned to a conventional 2-branch architecture.

6. The method according to claim 2, further comprising treating the seeds by one or more of the following treatments: cleaning, washing, drying, hydrating, priming, disinfecting, selecting for viability, coating, pelleting, applying a composition comprising at least one insecticidal, fungicidal, acaricidal, nematicidal compound, a plant growth regulator or a biological control agent.

7. The method according to claim 1, wherein Capsicum plants are cultivated plants of the species Capsicum annuum, plant lines of sweet pepper, or inbred lines of sweet pepper.

8. The method according to claim 3, wherein the first and the second parent plants are inbred lines and the seeds of Capsicum are F1 hybrid seeds.

9. The method according to claim 1, wherein the plants are pruned every 3, 4, 5, 6, 7 or 8 days.

10. The method according to claim 1, wherein the plants are pruned every 3, 4 or 5 days.

11. The method according to claim 1, further comprising removing the weakest or inward facing head or shoot at nodes that are pruned.

12. The method according to claim 1, where 4 branches are left after pruning.

13. The method according to claim 1, where 5 branches are left after pruning.

14. The method according to claim 1, wherein the plants are grown at a density of 10, 11, 12, 13, 14, 15, 16, 17, 18, 19, 20 or 21 branches per square meter.

15. The method according to claim 1, wherein the plants are inbred parent lines of a pepper type of: Bell (blocky), Lamuyo type, Dulce Italiano, Dulce California, Dulce Hungario, snack type pepper, conical, capia, sweet charleston, dolma, cherry, jalapeno, shakira, pencil or hot Charleston, Sivri, Hungarian wax, Kapya/Florinis, banana, Fresno, Serrano, Ancho, Anaheim, Pasilla, Santa Fe, Scotch bonnet, or Habanero.

16. The method according to claim 1, wherein the plurality of plants comprise at least 20, 30, 40, 50 or more plants of the same genotype.

17. A plurality of pruned Capsicum plants of an inbred parent line of cultivated pepper produced by claim 1.

18. A plurality of pruned Capsicum plants of an inbred parent line of cultivated pepper, wherein all the pruned plants have a 3-branch, 4-branch, 5-branch, 6-branch, 7-branch or 8-branch architecture.

19. The plurality of pruned Capsicum plants of claim 17, wherein at least one branch of each plant is supported with at least one vertical twine, string or rope.

20. The plurality of pruned Capsicum plants according to claim 17, wherein said plants are capable of producing an average mature seed yield per plant that is at least 10% higher than the average mature seed yield per plant produced by a plurality of pruned Capsicum plants of an inbred parent line of cultivated pepper having been pruned to the conventional 2-branch architecture.

21. The method according to claim 9, wherein the plants are pruned every 5 days.
